(19) United States
(12) Patent Application Publication
(10) Pub. No.: US 2020/0020424 A1
(43) Pub. Date: Jan. 16, 2020

(54) BLOCKCHAIN ELECTRONIC MEDICAL RECORD SYSTEM

(71) Applicant: Che-Min Lin, Taipei City (TW)

(72) Inventor: Che-Min Lin, Taipei City (TW)

(21) Appl. No.: 16/035,721

(22) Filed: Jul. 16, 2018

(51) Int. Cl. G16H 10/60; G16H 15/00; H04L 29/06; H04L 9/06

(52) U.S. Cl. CPC G16H 10/60; G16H 15/00; H04L 63/0823; H04L 2209/38; H04L 63/102; H04L 9/0637; H04L 63/083

(57) ABSTRACT

A blockchain electronic medical record system includes a hospital clinic end and patient end, a doctor at the hospital clinic end generating medical record data after the diagnosis of a patient, writing each of medical records into a connected computer to form electronic medical records, and storing the electronic medical records into a web server; the hospital clinic end giving the patient a secret key; the patient end obtaining the secret key, using the key to obtain an electronic medical record file from a space in connection with the web server and having a right to access to the electronic medical record file.

### (a) TECHNICAL FIELD OF THE INVENTION

The present invention relates to a blockchain electronic medical record (EMR) system, and more particularly to a system using a blockchain form to build up electronic medical records.

### (b) DESCRIPTION OF THE PRIOR ART

Conventionally, general patients' consultation data files are recorded through paperwork when they go to see a doctor of a hospital, and the doctor will handwrite the diagnosis results, prescription and remarks on the medical record form prepared by the hospital in which personal data is filled after diagnosis. Thereafter, the medical record form is archived and stored. When a patient goes back to see the doctor again, the hospital searches the medical record form again according to the patient's data and provides the doctor to complete the new content on it once again, which repeats again and again for future diagnoses. However, it is labor and time consumptive to make, search and file the medical record form and thus wasting the both parties' precious time. With the progress of software and hardware of information technologies, related applications, for example, electronation of paper medical records, are also developed in a medical field. Some doctors or clinics replace traditional paper medical records with electronic medical record; patients' medical record data built up by computer systems improves administrative efficiency and also perfecting medical care.

However, only a hospital computer filing the above computerized medical record data can read it, and patients themselves can even get their medical record only through the cumbersome application procedures of the hospital or even paying fees, causing the patients to be perplexed upon patient transfer or the undertaking of medical records. In some countries, although national health insurance IC card has a medical record in code form, the card itself is not designed to have detailed medical records, causing medical records unable to be fully grasped. Especially, patients themselves have no way to obtain their own medical records.

It can be seen that the circulation of the current medical data is still insufficient. Especially, patients even have less knowledge about their own medical records. Therefore, there are still unresolved problems in the conventional medical records of patients without effective solution at present.

### SUMMARY OF THE INVENTION

One object of the present invention is to provide a blockchain electronic medical record system, using a blockchain concept to access to electronic medical records.

To achieve the object mentioned above, the present invention proposes a blockchain electronic medical record system, including a hospital clinic end and patient end, a doctor at the hospital clinic end generating medical record data after the diagnosis of a patient, writing each of medical records into a connected computer to form electronic medical records, and storing the electronic medical records into a web server; the hospital clinic end giving the patient a secret key; the patient end obtaining the secret key, using the key to obtain an electronic medical record file from a space in connection with the web server and having a right to access to the electronic medical record file.

### BRIEF DESCRIPTION OF THE DRAWINGS

FIG. 1 is a schematically structural block diagram of a preferred embodiment of a blockchain electronic medical record system according to the present invention.

### DETAILED DESCRIPTION OF THE PREFERRED EMBODIMENTS

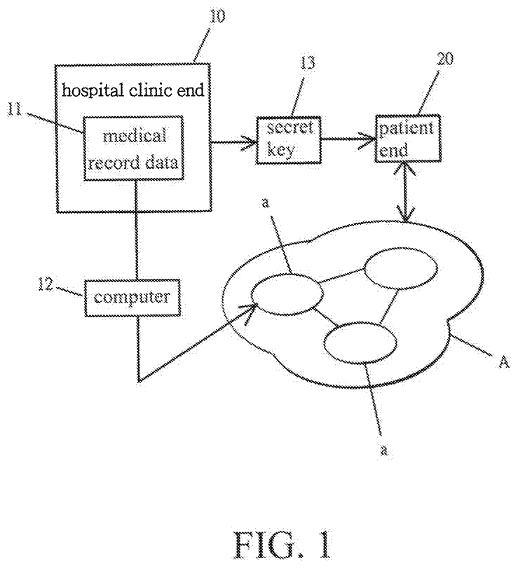

Referring to FIG. 1, a blockchain electronic medical record (EMR) system includes a hospital clinic end 10 and patient end 20.

At the hospital clinic end 10, a doctor generates various medical records 11 such as time, symptoms, conditions, medication, remarks and other messages after the diagnosis of a patient, and writes each record 11 after the diagnosis of the patient into a connected computer 12 to form electronic medical records and stores them in a block a in connection with a web server, which can be linked with the adjacent previous blocks a to form a blockchain A, which is a distributed database using cryptographic methods to generate data blocks relatively, and the medical content written in at the hospital clinic end 10 is generated into the data of a data block. Any data in the blockchain A can be obtained from every node in the blockchain A network because blockchain A is mostly open information so that the hospital clinic end 10 must add identification data of the node in relation to the record content in the data block, allowing the medical record content in the data block to be obtained only by the patient end 20 obtaining a secret key 13.

The patient end 20 can obtain the secret key 13 from the hospital clinic end 10 and use it to obtain the electronic medical record file from the blockchain A in connection with the web server. In addition, the patient end 20 has the right to access to the electronic medical record file.

With the above blockchain electronic medical record system, the data of the block or the block or electronic medical records cannot be arbitrarily tampered with, thereby keeping the completeness and safety of the medical records. The blockchain A and its blocks a can be built up on one or more than one web server, and each server is in connection with the other through a network.

The above connection refers to a wireless or wired network signal connection, and the electronic medical records any medical data of a patient such as diagnosis results, medication, treatment, surgical name, discharge, appendix, and other messages.

The hospital clinic end 10 may be any level of hospital, clinic, nursing center, nursing home, and the like which has the ability to generate electronic medical records.

The secret key 13 is a set of passwords. Specifically, the secret key 13 has a public key and private key, where the public key is a set of passwords and can be used as a credential to verify the private key after a certificate is issued through authentication, and the private key is a set of passwords, and must be kept by the patient end 20 and further verified with the public key to obtain the electronic medical record data.

The system structure of the present invention is simple, but the information is stable and safe, the data management and announcement are convenient, and the practical efficiency can be increased effectively. Especially, patients have the right to access to their own medical records, allowing the application thereof to be simple, convenient and safe.

The present invention has the following advantages over the prior art:
1. the hospital clinic end 10 stores patients' medical records in the blockchain A safely and completely, providing an innovative service and enhancing the contribution to medical qualities; and
2. patients have the ownership of their own medical records, capable of obtaining their own medical record data directly through network connection when needed and being better for self-health management.

I claim:

1. A blockchain electronic medical record system, comprising a hospital clinic end and patient end, a doctor at said hospital clinic end generating medical record data after the diagnosis of a patient, writing each of medical records into a connected computer to form electronic medical records, and storing said electronic medical records into a web server; said hospital clinic end giving said patient a secret key; said patient end obtaining said secret key, using said key to obtain an electronic medical record file from a space in connection with said web server and having a right to access to said electronic medical record file.

2. The system according to claim 1, wherein said hospital clinic end stores said electronic medical records into a block in connection with said web server, and said block is linked with adjacent blocks to establish a blockchain.
